(19) United States
(12) Patent Application Publication
(10) Pub. No.: US 2004/0180917 A1
(43) Pub. Date: Sep. 16, 2004

(54) Novel tricyclic dihydro-quinoline derivatives, method for preparing same and pharmaceutical compositions containing the same

(76) Inventors: Henri-Philippe Husson, Chevreuse (FR); Sylviane Giorgio-Renault, Paris (FR); Stephanie Desbene, Paris (FR); John Hickman, Paris (FR); Alain Pierre, Les Alluets Le Roi (FR); Laurence Kraus-Berthier, Colombes (FR); Bruno Pfeiffer, Saint Leu La Foret (FR); Pierre Renard, Le Chesnay (FR)

Correspondence Address: The Firm of Hueschen and Sage, 500 Columbia Plaza, 350 East Michigan Avenue, Kalamazoo, MI, 49007, US

(21) Appl. No.: 10/477,244

(22) PCT Filed: May 22, 2002
(86) PCT No.: PCT/FR02/01716

(30) Foreign Application Priority Data
May 23, 2001 (FR) 0106791

(51) Int. Cl. A61K 31/4745; C07D 498/02

(52) U.S. Cl. 514/291; 514/292; 546/80; 546/82

(57) ABSTRACT

A compound of formula (I):

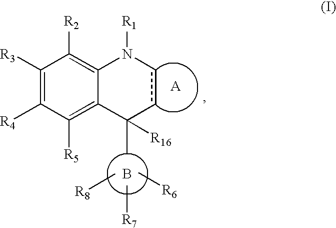

wherein:

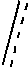

represents a single or double bond,

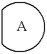

represents a ring system selected from

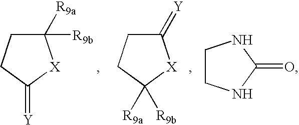

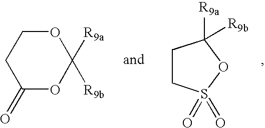

R₉ₐ, R_{9b}, R_{9c}, X and Y are as defined in the description,

R₁ represents a group selected from hydrogen, aryl, heteroaryl, cycloalkyl, optionally substituted alkyl, and COR₁₁, wherein R₁₁ is as defined in the description,

R₂ to R₈ each represent a group selected from hydrogen, halogen, hydroxy, polyhaloalkyl, nitro, optionally substituted alkyl, optionally substituted amino, optionally substituted alkoxy, —OPO(OH)₂ and

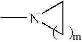

wherein m represents an integer such that 1≦m<4,

or R₂ with R₃, or R₃ with R₄, or R₄ with R₅, form, together with the carbon atoms carrying them, an optionally substituted, mono- or bi-cyclic group optionally containing 1 or 2 hetero atoms,

R₁₆ represents a hydrogen atom or an alkyl group,

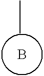

represents an aryl, heteroaryl or aryl-alkyl group,

its optical isomers, addition salts thereof with a pharmaceutically acceptable acid or base, and hydrates and solvates thereof.

The present invention relates to new tricyclic dihydroquinoline compounds, to a process for their preparation, to pharmaceutical compositions containing them and to their use as anti-cancer agents.

Anti-cancer therapeutic requirements call for the constant development of new anti-tumour agents with the aim of obtaining medicaments that are simultaneously more active and better tolerated.

Besides the fact that the compounds of the invention are new, they possess very valuable anti-tumour properties.

Compounds having a closely related structure have been described in the literature, especially furo[3,4-b]quinolin-1-one compounds as anti-osteoporotic agents (patent specification EP 0 634 169).

More specifically, the present invention relates to compounds of formula (I):

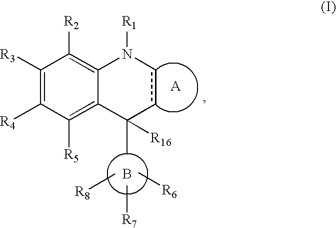

wherein:

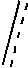

represents a single or double bond,

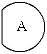

represents a ring system selected from

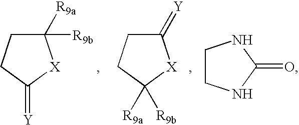

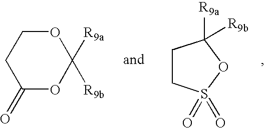

R₉ₐ, R_{9b} and R_{9c}, which may be the same or different, each represent a hydrogen atom or a linear or branched (C₁-C₆)alkyl group,

X represents an oxygen or sulphur atom or a group selected from CH₂, CH₂—CH₂, and NR_{9c} wherein R_{9c} represents a hydrogen atom or a linear or branched (C₁-C₆)alkyl group,

Y represents an oxygen or sulphur atom,

R₁ represents a hydrogen atom or a group selected from:

aryl,

heteroaryl,

(C₃-C₉)cycloalkyl,

linear or branched (C₁-C₆)alkyl optionally substituted by an aryl group, by a heteroaryl group, by a hydroxy group, by a linear or branched (C₁-C₆)alkoxy group, or by a group of formula NR₁₀ₐR_{10b} wherein R₁₀ₐ and R_{10b}, which may be the same or different, each represent a linear or branched (C₁-C₆)alkyl group optionally substituted by a hydroxy group or an amino group (itself optionally substituted by one or two linear or branched (C₁-C₆)alkyl groups), or R₁₀ₐ and R_{10b}, together with the nitrogen atom carrying them, form a nitrogen-containing heterocycle,

and COR₁₁ wherein R₁₁ represents a group:

aryl,

linear or branched (C₁-C₆)alkyl (optionally substituted by a group of formula NR₁₀ₐR_{10b} wherein R₁₀ₐ and R_{10b}, which may be the same or different, each represent a linear or branched (C₁-C₆)alkyl group optionally substituted by a hydroxy group or an amino group (itself optionally substituted by one or two linear or branched (C₁-C₆)alkyl groups), or R₁₀ₐ and R_{10b}, together with the nitrogen atom carrying them, form a nitrogen-containing heterocycle),

amino optionally substituted by one or more aryl groups, heteroaryl groups, or linear or branched (C₁-C₆)alkyl groups optionally substituted by a group of formula NR₁₀ₐR_{10b} wherein R₁₀ₐ and R_{10b}, which may be the same or different, each represent a linear or branched (C₁-C₆)alkyl group optionally substituted by a hydroxy group or an amino group (itself optionally substituted by one or two linear or branched (C₁-C₆)alkyl groups), or R₁₀ₐ and R_{10b}, together with the nitrogen atom carrying them, form a nitrogen-containing heterocycle,

or OR₁₂ wherein R₁₂ represents a hydrogen atom or an aryl group, or a linear or branched (C₁-C₆)alkyl group optionally substituted by a group of formula NR₁₀ₐR_{10b} wherein R₁₀ₐ and R_{10b}, which may be the same or different, each represent a linear or branched (C₁-C₆)alkyl group optionally substituted by a hydroxy group or an amino group (itself optionally substituted by one or two linear or branched (C₁-C₆)alkyl groups), or R₁₀ₐ and R_{10b}, together with the nitrogen atom carrying them, form a nitrogen-containing heterocycle,

R₂, R₃, R₄, R₅, R₆, R₇ and R₈, which may be the same or different, each represent:

a hydrogen atom,

a halogen atom,

a hydroxy group,

a linear or branched (C₁-C₆)polyhaloalkyl group,

a nitro group,

a linear or branched (C₁-C₆)alkyl group (optionally substituted by an amino group, itself optionally substituted by one or two linear or branched (C₁-C₆)alkyl groups),

an amino group optionally substituted by one or two linear or branched (C₁-C₆)alkyl groups, themselves optionally substituted by an amino group, a linear or branched (C₁-C₆)alkylamino group or a di-(C₁-C₆)alkylamino group in which the alkyl moieties may be linear or branched,

a linear or branched (C₁-C₆)alkoxy group optionally substituted by an amino group, a linear or branched (C₁-C₆)alkylamino group or a di-(C₁-C₆)alkylamino group in which the alkyl moieties may be linear or branched,

a group —OPO(OH)₂,

a group of formula

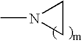

wherein m represents an integer such that 1≧m≧4,

or R₂ with R₃, or R₃ with R₄, or R₄ with R₅, form, together with the carbon atoms carrying them, a group of formula G:

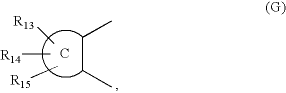

wherein

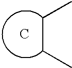

represents a 5- to 12-membered, mono- or bi-cyclic, aromatic or non-aromatic group optionally containing 1 or 2 hetero atoms selected from O, S and N, and R₁₃, R₁₄ and R₁₅, which may be the same or different, each represent a hydrogen atom or halogen atom or a group selected from linear or branched (C₁-C₆)alkyl (optionally substituted by an amino group, itself optionally substituted by one or two linear or branched (C₁-C₆)alkyl groups), hydroxy, linear or branched (C₁-C₆)alkoxy (optionally substituted by an amino group, a linear or branched (C₁-C₆)alkylamino group or a di-(C₁-C₆)alkylamino group in which the alkyl moieties may be linear or branched), linear or branched (C₁-C₆)polyhaloalkyl, amino (optionally substituted by one or more linear or branched (C₁-C₆)alkyl groups, themselves optionally substituted by an amino group, a linear or branched (C₁-C₆)alkylamino group or a di-(C₁-C₆)alkylamino group in which the alkyl moieties may be linear or branched), nitro, —OPO(OH)₂, linear or branched (C₁-C₆)acyl and (C₁-C₂)alkylenedioxy groups,

R₁₆ represents a hydrogen atom or a linear or branched (C₁-C₆)alkyl group,

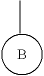

represents an aryl group, heteroaryl group or aryl-(C₁-C₆)alkyl group wherein the alkyl moiety is linear or branched,

to their optical isomers, to addition salts thereof with a pharmaceutically acceptable acid or base, and to hydrates and solvates thereof,

with the proviso that at least one of the groups R₂ to R₈ represents a linear or branched (C₁-C₆)aminoalkyl group (optionally N-substituted by one or two linear or branched (C₁-C₆)alkyl groups), an alkylaminoalkylamino group wherein alkyl is (C₁-C₆) and is linear or branched, a dialkylaminoalkylamino group wherein alkyl is (C₁-C₆) and is linear or branched, an alkylaminoalkoxy group wherein alkyl is (C₁-C₆) and is linear or branched, a dialkylaminoalkoxy group wherein alkyl is (C₁-C₆) and is linear or branched or a group —OPO(OH)₂, or R₂ with R₃, or R₃ with R₄, or R₄ with R₅ form, together with the carbon atoms carrying them, a group of formula G wherein at least one of the groups R₁₃ to R₁₅ represents a linear or branched (C₁-C₆)aminoalkyl group (optionally N-substituted by one or two linear or branched (C₁-C₆)alkyl groups), an alkylaminoalkylamino group wherein alkyl is (C₁-C₆) and is linear or branched, a dialkylaminoalkylamino group wherein alkyl is (C₁-C₆) and is linear or branched, an alkylaminoalkoxy group wherein alkyl is (C₁-C₆) and is linear or branched, a dialkylaminoalkoxy group wherein alkyl is (C₁-C₆) and is linear or branched, hydroxy or a group —OPO(OH)₂.

Among the pharmaceutically acceptable acids there may be mentioned, without implying any limitation, hydrochloric acid, hydrobromic acid, sulphuric acid, phosphoric acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, benzenesulphonic acid, camphoric acid etc.

Among the pharmaceutically acceptable bases there may be mentioned, without implying any limiation, sodium hydroxide, potassium hydroxide, triethylamine, tert-butylamine.

An aryl group is understood to mean phenyl, biphenylyl, naphthyl or tetrahydronaphthyl, each of those groups being optionally substituted by one or more identical or different atoms or groups selected from halogen atoms and linear or branched (C₁-C₆)alkyl, hydroxy, linear or branched (C₁-C₆)alkoxy, linear or branched (C₁-C₆)polyhaloalkyl, amino (optionally substituted by one or more linear or branched (C₁-C₆)alkyl groups), nitro, linear or branched (C₁-C₆)acyl and (C₁-C₂)alkylenedioxy groups.

A heteroaryl group is understood to mean a 5- to 12-membered, mono- or bi-cyclic, aromatic group containing one, two or three hetero atoms selected from oxygen, nitrogen and sulphur, it being understood that the heteroaryl group may be optionally substituted by one or more identical or different atoms or groups selected from halogen atoms and linear or branched (C₁-C₆)alkyl groups, hydroxy groups, linear or branched (C₁-C₆)alkoxy groups, linear or branched (C₁-C₆)polyhaloalkyl groups and amino groups (optionally substituted by one or more linear or branched (C₁-C₆)alkyl groups). Among the heteroaryl groups, there may be mentioned, without implying any limitation, the groups thienyl, pyridyl, furyl, pyrrolyl, imidazolyl, oxazolyl, isoxazolyl, thiazolyl, isothiazolyl, quinolyl, isoquinolyl and pyrimidinyl.

A nitrogen-containing heterocycle is understood to mean a 5- to 7-membered, monocyclic, saturated group containing one, two or three hetero atoms, one of those hetero atoms being the nitrogen atom and the additional hetero atom(s) optionally present being selected from oxygen, nitrogen and sulphur atoms. Preferred nitrogen-containing heterocycles are the groups pyrrolidinyl, piperidyl, morpholinyl or piperazinyl.

Among the groups of formula G, there may be mentioned, without implying any limitation, those wherein

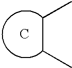

represents a phenylene, naphthylene or cyclopentenylene group, and groups of formulae G₁ to G₅:

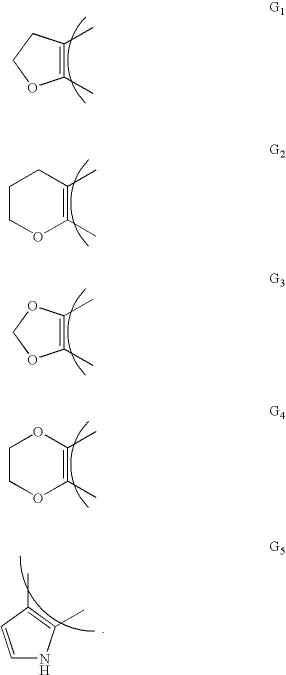

Preferred compounds of formula (I) are those wherein [symbol] represents a double bond.

An advantageous aspect of the invention relates to compounds of formula (I) wherein R₂ to R₈, which may be the same or different, each represent a group selected from hydrogen, linear or branched (C₁-C₆)alkoxy (optionally substituted by an amino group, linear or branched (C₁-C₆)alkylamino group or di-(C₁-C₆)alkylamino group wherein the alkyl moieties may be linear or branched) and OPO(OH)₂.

Another advantageous aspect of the invention relates to compounds of formula (I) wherein R₂ and R₃, or R₃ and R₄, form, together with the carbon atoms carrying them, a group of formula G.

Among those compounds, special preference is given to those wherein R₃ and R₄, together with the carbon atoms carrying them, form a group of formula G₃ or G₄ as defined hereinbefore, and those wherein R₂ and R₃, together with the carbon atoms carrying them, form a group of formula G wherein

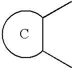

represents a phenylene group, and R₁₃, R₁₄ and R₁₅, which may be the same or different, each represent a group selected from hydrogen, hydroxy, linear or branched (C₁-C₆)alkoxy (optionally substituted by an amino group, linear or branched (C₁-C₆)alkylamino group or di-(C₁-C₆)alkylamino group wherein the alkyl moieties may be linear or branched) and OPO(OH)₂.

An advantageous aspect of the invention relates to compounds of formula (I) wherein R₁₆ represents a hydrogen atom.

Another advantageous aspect of the invention relates to compounds of formula (I) wherein R₁₆ represents a linear or branched (C₁-C₆)alkyl group.

An advantageous aspect of the invention relates to compounds of formula (I) wherein

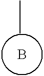

represents an aryl group. Among those compounds, special preference is given to those wherein

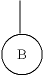

represents a phenyl group.

Another advantageous aspect of the invention relates to compounds of formula (I) wherein

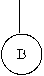

represents a heteroaryl group.

Preferred compounds of formula (I) are those wherein

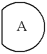

represents a ring system

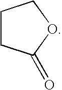

Preferred compounds of formula (I) are those wherein R₁ represents a hydrogen atom or a linear or branched (C₁-C₆)alkyl group.

Among the compounds of the invention there may be mentioned more specifically:

2-hydroxy-7-(3,4,5-trimethoxyphenyl)-7,11-dihydrobenzo[h]furo[3,4-b]quinolin-8(10H)-one and its optical isomers,

and 8-oxo-7-(3,4,5-trimethoxyphenyl)-7,8,10,11-tetrahydrobenzo[h]furo[3,4-b]-quinolin-2-yl dihydrogen phosphate, its disodium salt, and optical isomers thereof.

The invention relates also to a process for the preparation of compounds of formula I, which process is characterised in that a compound of formula (II):

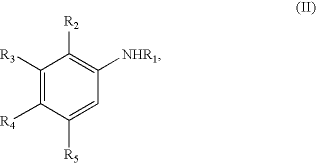

wherein R₁, R₂, R₃, R₄ and R₅ are as defined for formula (I),

is reacted with a compound of formula (IV):

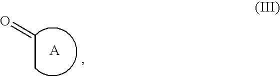

wherein

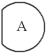

is as defined for formula (I),

and with a compound of formula (IV):

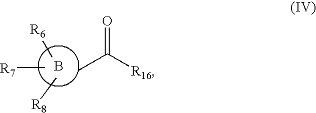

wherein

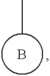

R₆, R₇, R₈ and R₁₆ are as defined for formula (I),

to yield the compound of formula (Ia), a particular case of the compounds of formula (I)

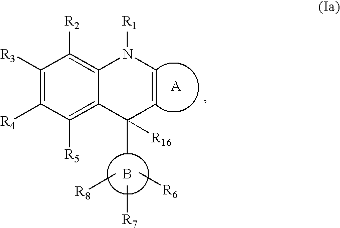

wherein

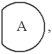

R₁, R₂, R₃, R₄, R₅, R₆, R₇, R₈, R₁₆ and

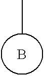

are as defined hereinbefore,

which, if desired, is reduced to yield the compound of formula (Ib), a particular case of the compounds of formula (I):

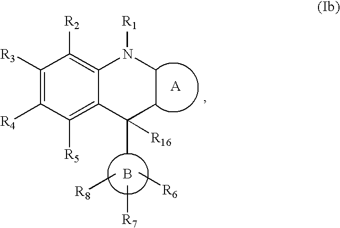

wherein

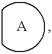

R₁, R₂, R₃, R₄, R₅, R₆, R₇, R₈, R₁₆ and

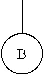

are as defined hereinbefore,

the compounds of formulae (Ia) and (Ib) constituting the totality of the compounds of formula (I), which are purified, if necessary, according to a conventional purification technique, are separated, if desired, into their optical isomers according to a conventional separation technique and are converted, if desired, into their addition salts with a pharmaceutically acceptable acid or base.

The compounds of formula (I) wherein

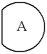

contains a thioxo (═S) group may also be obtained by thionation of the corresponding oxo (═O) group.

The compounds of formula (I) wherein at least one of the groups R₂ to R₈ represents a group —OPO(OH)₂, or R₂ with R₃, or R₃ with R₄, or R₄ with R₅, form, together with the carbon atoms carrying them, a group of formula G wherein at least one of the groups R₁₃ to R₁₅ represents a group —OPO(OH)₂ may also be obtained starting from the corresponding alcohol.

Besides the fact that the compounds of the present invention are new, they possess valuable pharmacological properties. They have cytotoxic properties that make them useful in the treatment of cancers.

The invention relates also to pharmaceutical compositions comprising as active ingredient at least one compound of formula (I) together with one or more inert, non-toxic, appropriate excipients. Among the pharmaceutical compositions according to the invention there may be mentioned more especially those that are suitable for oral, parenteral (intravenous, intramuscular or subcutaneous) or nasal administration, tablets or dragees, sublingual tablets, gelatin capsules, lozenges, suppositories, creams, ointments, dermal gels, injectable preparations, drinkable suspensions etc.

The useful dosage can be varied according to the nature and severity of the disorder, the administration route and also the age and weight of the patient and any associated treatments. The dosage varies from 0.5 mg to 2 g per 24 hours in one or more administrations.

The following Examples illustrate the invention but do not limit it in any way.

The starting materials used are known compounds or prepared according to known methods of preparation.

Preparations A to G yield synthesis intermediates that are useful in the preparation of compounds of the invention.

The structures of the compounds described in the Examples have been determined in accordance with the customary spectrometric techniques (infrared, NMR, mass spectrometry).

Preparation A: 3-(2-Dimethylaminoethoxy)-benzaldehyde

To 10 mmol of sodium methanolate dissolved in isopropanol there are added 10 mmol of 3-hydroxybenzaldehyde dissolved in isopropanol. The reaction mixture is then stirred for 30 minutes at ambient temperature; 10 mmol of N,N-dimethyl-2-chloroethylamine dissolved in toluene are then added and the mixture is heated at reflux. After reacting for 6 hours, the solvents are evaporated off and the residue obtained is then taken up in water and extracted with ether. The combined organic phases are then dried and evaporated, and the residue obtained is purified by chromatography over silica to yield the expected product.

Preparation B: 2-(2-Dimethylaminoethoxy)-3-methoxyaniline

The expected product is obtained according to the procedure described in Preparation A, starting from 2-amino-6-methoxyphenol (the preparation of which is described in J. Org. Chem. 1926, 2007) and N,N-dimethyl-2-chloroethylamine.

Preparation C: 8-(3-Diethylaminopropoxy)-1-naphthylamine

The expected product is obtained according to the procedure described in Preparation A, starting from 8-amino-2-naphthol and N,N-diethyl-3-chloropropylamine.

Preparation D: 8-Amino-2-naphthyl dibenzyl phosphate

To 10 mmol of 8-amino-2-naphthol dissolved in a 50/50 mixture of acetonitrile and dimethylformamide there are added, at −10° C., 50 mmol of bromotrichloromethane, 20 mmol of triethylamine and 1 mmol of 4-dimethylaminopyridine and then, 1 minute later, 10 mmol of dibenzyl phosphite dropwise, whilst maintaining the temperature of the reaction mixture at −10° C.

An aqueous solution of potassium phosphate is then added and the mixture is then extracted with ethyl acetate. The combined organic phases are washed and then dried and evaporated to yield the expected product.

Preparation E: 2-Amino-6-methoxyphenyl dibenzyl phosphate

The expected product is obtained according to the procedure described in Preparation D, starting from 2-amino-6-methoxyphenol.

Preparation F: 3-(2-Diethylaminoethoxy)-aniline

The expected product is obtained according to the procedure described in Preparation A, starting from 3-aminophenol and N,N-diethyl-2-chloroethylamine.

Preparation G: 3-(2-Diethylaminoethoxy)-benzaldehyde

The expected product is obtained according to the procedure described in Preparation A, starting from 3-hydroxybenzaldehyde and N,N-diethyl-2-chloroethylamine.

### EXAMPLE 1

### 9-[3-(2-Dimethylaminoethoxy)-phenyl]-6,7-methylenedioxy-4,9-dihydrofuro[3,4-b]quinolin-1(3,H)-one

To 10 mmol of 3,4-methylenedioxy-aniline dissolved in ethanol there are added 10 mmol of tetronic acid and 10 mmol of the compound described in Preparation A, and the reaction mixture is then heated at reflux. After cooling, the precipitate obtained is filtered off and then washed and recrystallised to yield the expected product.

### EXAMPLE 2

### 5-(2-Dimethylaminoethoxy)-6-methoxy-9-(3,4,5-trimethoxy-phenyl)-4,9-dihydrofuro [3,4-b]quinolin-1 (3H)-one

The expected product is obtained according to the procedure described in Example 1, starting from tetronic acid, 3,4,5-trimethoxybenzaldehyde and the compound described in Preparation B.

### EXAMPLE 3

### 2-(3-Diethylaminopropoxy)-7-(3,4,5-trimethoxyphenyl)-7,11-dihydrobenzo[h]furo[3,4-b]quinolin-8(10H)-one

The expected product is obtained according to the procedure described in Example 1, starting from tetronic acid, 3,4,5-trimethoxybenzaldehyde and the compound described in Preparation C.

### EXAMPLE 4

### 2-Hydroxy-7-(3,4,5-trimethoxyphenyl)-7,11-dihydrobenzo [h]furo-[3,4-blquinolin-8(10H)-one:

The expected product is obtained according to the procedure described in Example 1, starting from tetronic acid, 3,4,5-trimethoxybenzaldehyde and 8-amino-2-naphthol.

Melting point: >260° C.

### EXAMPLE 5

### 8-Oxo-7-(3,4,5-trimethoxyphenyl)-7,8,10,11-tetrahydrobenzolh]-furo[3,4-b]quinolin-2-yl dihydrogen phosphate

Step A: Dibenzyl 8-oxo-7(3,4,5-trimethoxyphenyl)-7,8,10,11-tetrahydro-benzo[h]furo[3,4-b]quinolin-2-yl phosphate

The expected product is obtained according to the procedure described in Preparation D, starting from the compound of Example 4.

Step B: 8-Oxo-7-(3,4,5-trimethoxyphenyl)-7,8,10,11-tetrahydrobenzo[h]furo-[3,4-b]quinolin-2-yl dihydrogen phosphate

The product obtained in the previous Step is dissolved in ethanol and then hydrogenated in the presence of 5% palladium-on-carbon, under 1 atmosphere and with stirring. The mixture is then filtered over Celite and the solvent is then evaporated off from the filtrate. The residue obtained is purified by chromatography over silica to yield the expected product.

Melting point of the disodium salt: 270 to 280° C.

### EXAMPLE 6

### 6-(2-Diethylaminoethoxy)-9-(3,4,5-trimethoxyphenyl)-4,9-dihydrofuro[3,4-b]quinolin-1(3H)-one

The expected product is obtained according to the procedure described in Example 1, starting from tetronic acid, 3,4,5-trimethoxybenzaldehyde and the compound described in Preparation F.

Melting point: 205° C.

### EXAMPLE 7

### 6-Methoxy-1-oxo-9-(3,4,5-trimethoxyphenyl)-1,3,4,9-tetrahydro-furo[3,4-b]quinolin-5-yl dihydrogen phosphate

The expected product is obtained according to the procedure described in Example 5, starting from tetronic acid, 3,4,5-trimethoxybenzaldehyde and the compound described in Preparation E.

### EXAMPLE 8

### 9-[3-(2-Diethylaminoethoxy)-phenyl]-6,7-methylenedioxy-4,9-dihydrofuro[3,4-b]quinolin-1(3H)-one hydrochloride

The expected product is obtained according to the procedure described in Example 1, replacing the compound of Preparation A by the compound of Preparation G.

Melting point: >205° C.

### EXAMPLE 9

### 9-[3-(2-Diethylaminoethoxy)-phenyl]-6-methoxy-4,9-dihydrofuro-[3,4-b]quinolin-1(3H)-one hydrochloride:

The expected product is obtained according to the procedure described in Example 1, starting from tetronic acid, 3-methoxyaniline and the compound described in Preparation G.

Melting point: >205° C.

### EXAMPLE 10

### 6-(2-Diethylaminoethoxy)-9-[3-(2-diethylaminoethoxy)-phenyl]-4,9-dihydrofuro[3,4-b]quinolin-1(3H)-one dihydrochloride

The expected product is obtained according to the procedure described in Example 1, starting from tetronic acid, the compound described in Preparation F and the compound described in Preparation G.

Melting point: >205° C.

### EXAMPLE 11

### 3-(4-Methyl-6,7-methylenedioxy-1-oxo-1,3,4,9-tetrahydrofuro-[3,4-b]quinolin-9-yl)-phenyl dihydrogen phosphate

Step A: N-methyl-3,4-methylenedioxyaniline

A solution of 3,4-methylenedioxyaniline (10 mmol) in formic acid (36 ml) is heated at reflux for 1 hour. After removing the excess formic acid, the residue obtained is diluted with water and then extracted with dichloromethane. The combined organic phases are evaporated. The residue obtained is dissolved in ether, and then lithium aluminium hydride (42 mmol) is added at 10° C., in small portions. After stirring for 3 hours at ambient temperature, water and 10% sodium hydroxide solution are added and the mixture is then filtered over Celite. The filtrate is dried and evaporated and the residue obtained is purified by chromatography over silica, using dichloromethane as eluant, to yield the expected product in the form of an oil.

Step B: 9-(3-Hydroxyphenyl)-4-methyl-6,7-methylenedioxy-4,9-dihydrofuro[3,4-b]-quinolin-1 (3H)-one

The expected product is obtained according to the procedure described in Example 1, starting from tetronic acid, 3-hydroxybenzaldehyde and the compound obtained in the Step above.

Step C: Dibenzyl 3-(4-methyl-6,7-methylenedioxy-1-oxo-1,3,4,9-tetrahydrofuro-[3,4-b]quinolin-9-yl)-phenyl phosphate

The expected product is obtained according to the procedure described in Preparation D, starting from the compound obtained in the Step above.

Step D: 3-(4-Methyl-6,7-methylenedioxy-1-oxo-1,3,4,9-tetrahydrofuro[3,4-b]-quinolin-9-yl)-phenyl dihydrogen phosphate

The expected product is obtained according to the procedure described in Step B of Example 5, starting from the compound obtained in the Step above.

### EXAMPLE 12

### 3-(9-Methyl-6,7-methylenedioxy-1-oxo-1,3,4,9-tetrahydrofuro[3,4-b]-quinolin-9-yl)-phenyl dihydrogen phosphate

Step A: 9-(3-Hydroxyphenyl)-9-methyl-6,7-methylenedioxy-4,9-dihydrofuro[3,4-b]-quinolin-1 (3H)-one

100 mmol of 3-hydroxy-acetophenonone are added to 10 mmol of tetronic acid dissolved in trifluoroacetic acid, and the reaction mixture is then heated at reflux for 3 hours. Then, 10 mmol of 3,4-methylenedioxyaniline are added and the solution is then heated at reflux for 2 hours 30 minutes. After evaporating off the solvent under reduced pressure, the residue obtained is purified by chromatography over silica (eluant:dichloromethane/ethyl acetate 90/10) to yield the expected product.

Step B: Dibenzyl 3-(9-methyl-6,7-methylenedioxy-1-oxo-1,3,4,9-tetrahydrofuro[3,4-b]quinolin-9-yl)-phenyl phosphate

The expected product is obtained according to the procedure described in Preparation D, starting from the compound obtained in the Step above.

Step C: 3-(9-Methyl-6,7-methylenedioxy-1-oxo-1,3,4,9-tetrahydrofuro[3,4-b]-quinolin-9-yl)-phenyl dihydrogen phosphate

The expected product is obtained according to the procedure described in Step B of Example 5, starting from the compound obtained in the Step above.

Pharmacological Study of Compounds of the Invention

### EXAMPLE 13

### In Vitro Cytotoxicity

Three cell lines were used:

1 murine leukaemia, L11210,

1 human non-small-cell lung carcinoma, A549,

1 human colon carcinoma, HT29.

The cells are cultured in RPMI 1640 complete culture medium containing 10% foetal calf serum, 2 mM glutamine, 50 units/ml of penicillin, 50 μg/ml of streptomycin and 10 mM Hepes, pH=7.4. The cells are distributed on microplates and are exposed to the cytotoxic compounds. The cells are then incubated for 2 days (L1210) or 4 days (A549, HT29). The number of viable cells is then quantified by a colorimetric assay, the Microculture Tetrazolium Assay (Cancer Res. 1987, 47, 939-942).

The results obtained show that the compounds of the invention have in vitro cytotoxicity.

By way of example, the compound of Example 4 has an IC₅₀ (concentration of cytotoxic agent which inhibits proliferation of the treated cells by 50%) of 7 nM (L1210).

### EXAMPLE 14

### Pharmaceutical Composition

Formula for the preparation of 1000 tablets each
containing 10 mg of active ingredient

| | |
|---|---|
| Compound of Example 4 | 10 g |
| Hydroxypropyl cellulose | 2 g |
| Wheat starch | 10 g |
| Lactose | 100 g |
| Magnesium stearate | 3 g |
| Talc | 3 g |

1. Compound of formula (I): 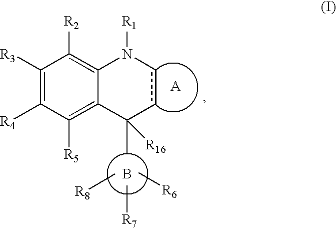
wherein: 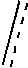
represents a single or double bond, 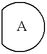
represents a ring system selected from 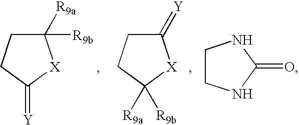 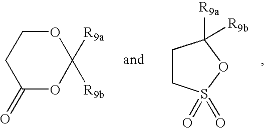
R₉ₐ, R_{9b} and R_{9c}, which may be the same or different, each represent a hydrogen atom or a linear or branched (C₁-C₆)alkyl group,
X represents an oxygen or sulphur atom or a group selected from CH₂, CH₂—CH₂, and NR_{9c} wherein R_{9c}, represents a hydrogen atom or a linear or branched (C₁-C₆)alkyl group,
Y represents an oxygen or sulphur atom,
R₁ represents a hydrogen atom or a group selected from:
aryl,
heteroaryl,
(C₃-C₈)cycloalkyl,
linear or branched (C₁-C₆)alkyl optionally substituted by an aryl group, by a heteroaryl group, by a hydroxy group, by a linear or branched (C₁-C₆)alkoxy group, or by a group of formula NR₁₀ₐR_{10b} wherein R₁₀ₐ and R_{10b}, which may be the same or different, each represent a linear or branched (C₁-C₆)alkyl group optionally substituted by a hydroxy group or an amino group (itself optionally substituted by one or two linear or branched (C₁-C₆)alkyl groups), or R₁₀ₐ and R_{10b}, together with the nitrogen atom carrying them, form a nitrogen-containing heterocycle,
and COR₁₁ wherein R₁₁ represents a group:
aryl,
linear or branched (C₁-C₆)alkyl (optionally substituted by a group of formula NR₁₀ₐR_{10b} wherein R₁₀ₐ and R_{10b}, which may be the same or different, each represent a linear or branched (C₁-C₆)alkyl group optionally substituted by a hydroxy group or an amino group (itself optionally substituted by one or two linear or branched (C₁-C₆)alkyl groups), or R₁₀ₐ and R_{10b}, together with the nitrogen atom carrying them, form a nitrogen-containing heterocycle),
amino optionally substituted by one or more aryl groups, heteroaryl groups, or linear or branched (C₁-C₆)alkyl groups optionally substituted by a group of formula NR₁₀ₐR_{10b} wherein R₁₀ₐ and R_{10b}, which may be the same or different, each represent a linear or branched (C₁-C₆)alkyl group optionally substituted by a hydroxy group or an amino group (itself optionally substituted by one or two linear or branched (C₁-C₆)alkyl groups), or R₁₀ₐ and R_{10b}, together with the nitrogen atom carrying them, form a nitrogen-containing heterocycle,
or OR₁₂ wherein R₁₂ represents a hydrogen atom or an aryl group, or a linear or branched (C₁-C₆)alkyl group optionally substituted by a group of formula NR₁₀ₐR_{10b} wherein R₁₀ₐ and R_{10b}, which may be the same or different, each represent a linear or branched (C₁-C₆)alkyl group optionally substituted by a hydroxy group or an amino group (itself optionally substituted by one or two linear or branched (C₁-C₆)alkyl groups), or R₁₀ₐ and R_{10b}, together with the nitrogen atom carrying them, form a nitrogen-containing heterocycle,
R₂, R₃, R₄, R₅, R₆, R₇ and R₈, which may be the same or different, each represent:
a hydrogen atom,
a halogen atom,
a hydroxy group,
a linear or branched (C₁-C₆)polyhaloalkyl group,
a nitro group,
a linear or branched (C₁-C₆)alkyl group (optionally substituted by an amino group, itself optionally substituted by one or two linear or branched (C₁-C₆)alkyl groups),
an amino group optionally substituted by one or two linear or branched (C₁-C₆)alkyl groups, themselves optionally substituted by an amino group, a linear or branched (C₁-C₆)alkylamino group or a di-(C₁-C₆)alkylamino group in which the alkyl moieties may be linear or branched,
a linear or branched (C₁-C₆)alkoxy group optionally substituted by an amino group, a linear or branched (C₁-C₆)alkylamino group or a di-(C₁-C₆)alkylamino group in which the alkyl moieties may be linear or branched,
a group —OPO(OH)₂,
a group of formula 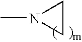
wherein m represents an integer such that 1≦m≦4,
or R₂ with R₃, or R₃ with R₄, or R₄ with R₅, form, together with the carbon atoms carrying them, a group of formula G: 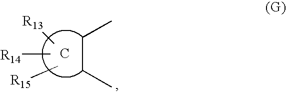
wherein 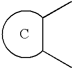
represents a 5- to 12-membered, mono- or bi-cyclic, aromatic or non-aromatic group optionally containing 1 or 2 hetero atoms selected from O, S and N, and R₁₃, R₁₄ and R₁₅, which may be the same or different, each represent a hydrogen atom or halogen atom or a group selected from linear or branched (C₁-C₆)alkyl (optionally substituted by an amino group, itself optionally substituted by one or two linear or branched (C₁-C₆)alkyl groups), hydroxy, linear or branched (C₁-C₆)alkoxy (optionally substituted by an amino group, a linear or branched (C₁-C₆)alkylamino group or a di-(C₁-C₆)alkylamino group in which the alkyl moieties may be linear or branched), linear or branched (C₁-C₆)polyhaloalkyl, amino (optionally substituted by one or more linear or branched (C₁-C₆)alkyl groups, themselves optionally substituted by an amino group, a linear or branched (C₁-C₆)alkylamino group or a di-(C₁-C₆)alkylamino group in which the alkyl moieties may be linear or branched), nitro, —OPO(OH)₂, linear or branched (C₁-C₆)acyl and (C₁-C₂)alkylenedioxy groups,
R₁₆ represents a hydrogen atom or a linear or branched (C₁-C₆)alkyl group, 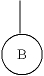
represents an aryl group, heteroaryl group or aryl-(C₁-C₆)alkyl group wherein the alkyl moiety is linear or branched,
its optical isomers, addition salts thereof with a pharmaceutically acceptable acid or base, and hydrates and solvates thereof,
with the proviso that at least one of the groups R₂ to R₈ represents a linear or branched (C₁-C₆)aminoalkyl group (optionally N-substituted by one or two linear or branched (C₁-C₆)alkyl groups), an alkylaminoalkylamino group wherein alkyl is (C₁-C₆) and is linear or branched, a dialkylaminoalkylamino group wherein alkyl is (C₁-C₆) and is linear or branched, an alkylaminoalkoxy group wherein alkyl is (C₁-C₆) and is linear or branched, a dialkylaminoalkoxy group wherein alkyl is (C₁-C₆) and is linear or branched or a group —OPO(OH)₂, or R₂ with R₃, or R₃ with R₄, or R₄ with R₅ form, together with the carbon atoms carrying them, a group of formula G wherein at least one of the groups R₁₃ to R₁₅ represents a linear or branched (C₁-C₆)aminoalkyl group (optionally N-substituted by one or two linear or branched (C₁-C₆)alkyl groups), an alkylaminoalkylamino group wherein alkyl is (C₁-C₆) and is linear or branched, a dialkylaminoalkylamino group wherein alkyl is (C₁-C₆) and is linear or branched, an alkylaminoalkoxy group wherein alkyl is (C₁-C₆) and is linear or branched, a dialkylaminoalkoxy group wherein alkyl is (C₁-C₆) and is linear or branched, hydroxy or a group —OPO(OH)₂,
an aryl group being understood to mean phenyl, biphenylyl, naphthyl or tetrahydronaphthyl, each of those groups being optionally substituted by one or more identical or different atoms or groups selected from halogen atoms and linear or branched (C₁-C₆)alkyl, hydroxy, linear or branched (C₁-C₆)alkoxy, linear or branched (C₁-C₆)polyhaloalkyl, amino (optionally substituted by one or more linear or branched (C₁-C₆)alkyl groups), nitro, linear or branched (C₁-C₆)acyl and (C₁-C₂)alkylenedioxy groups,
a heteroaryl group being understood to mean a 5- to 12-membered, mono- or bi-cyclic, aromatic group containing one, two or three hetero atoms selected from oxygen, nitrogen and sulphur, it being understood that the heteroaryl group may be optionally substituted by one or more identical or different atoms or groups selected from halogen atoms and linear or branched (C₁-C₆)alkyl groups, hydroxy groups, linear or branched (C₁-C₆)alkoxy groups, linear or branched (C₁-C₆)polyhaloalkyl groups and amino groups (optionally substituted by one or more linear or branched (C₁-C₆)alkyl groups),
and a nitrogen-containing heterocycle being understood to mean a 5- to 7-membered, monocyclic, saturated group containing one, two or three hetero atoms, one of those hetero atoms being the nitrogen atom and the additional hetero atom(s) optionally present being selected from oxygen, nitrogen and sulphur atoms.

2. A compound of formula (I) according to claim 1, wherein 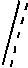
represents a double bond.

3. A compound of formula (I) according to either claim 1 or claim 2, wherein R₂ and R₃, or R₃ and R₄, together with the carbon atoms carrying them, form a group of formula G: 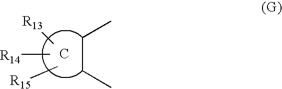
wherein 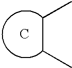
represents a 5- to 12-membered, mono- or bi-cyclic, aromatic or non-aromatic group optionally containing 1 or 2 hetero atoms selected from O, S and N, and R₁₃, R₁₄ and R₁₅, which may be the same or different, each represent a hydrogen 5 atom or halogen atom or a group selected from linear or branched (C₁-C₆)alkyl (optionally substituted by an amino group, itself optionally substituted by one or two linear or branched (C₁-C₆)alkyl groups), hydroxy, linear or branched (C₁-C₆)alkoxy (optionally substituted by an amino group, a linear or branched (C₁-C₆)alkylamino group or a di-(C₁-C₆)alkylamino group in which the alkyl moieties may be linear or branched), linear or branched (C₁-C₆)polyhaloalkyl, amino (optionally substituted by one or more linear or branched (C₁-C₆)alkyl groups, themselves optionally substituted by an amino group, a linear or branched (C₁-C₆)alkylamino group or a di-(C₁-C₆)alkylamino group in which the alkyl moieties may be linear or branched), nitro, —OPO(OH)₂, linear or branched (C₁-C₆)acyl and (C₁-C₂)alkylenedioxy groups.

4. A compound of formula (I) according to claim 3, wherein R₃ and R₄, together with the carbon atoms carrying them, form a group of formula G₃ or G₄:

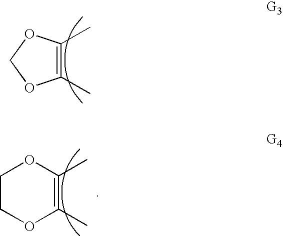

5. A compound of formula (I) according to claim 3, wherein R₂ and R₃, together with the carbon atoms carrying them, form a group of formula G wherein 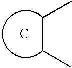
represents a phenylene group, and R₁₃, R₁₄ and R₁₅, which may be the same or different, each represent a group selected from hydrogen, hydroxy, linear or branched (C₁-C₆)alkoxy (optionally substituted by an amino group, linear or branched (C₁-C₆)alkylamino group or di-(C₁-C₆)alkylamino group wherein the alkyl moieties may be linear or branched) and OPO(OH)₂.

6. A compound of formula (I) according to any one of claims 1 to 5, wherein R₁₆ represents a hydrogen atom.

7. A compound of formula (I) according to any one of claims 1 to 5, wherein R₁₆ represents a (C₁-C₆)alkyl group.

8. A compound of formula (I) according to any one of claims 1 to 7, wherein 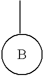
represents an aryl group.

9. A compound of formula I according to claim 8, wherein 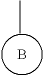
represents a phenyl group.

10. A compound of formula (I) according to any one of claims 1 to 7, wherein 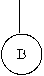
represents a heteroaryl group.

11. A compound of formula (I) according to any one of claims 1 to 10, wherein 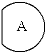
represents a ring system 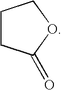
.

12. A compound of formula (I) according to any one of claims 1 to 11, wherein R₁ represents a hydrogen atom or a (C₁-C₆)alkyl group.

13. A compound of formula (I) according to claim 1, which is 2-hydroxy-7-(3,4,5-trimethoxyphenyl)-7,1′-dihydrobenzo[h]furo[3,4-b]quinolin-8(10H)-one and its optical isomers.

14. A compound of formula (I) according to claim 1, which is 8-oxo-7-(3,4,5-trimethoxyphenyl)-7,8,10,11-tetrahydrobenzo[h]furo[3,4-b]quinolin-2-yl dihydrogen phosphate, its disodium salt, or optical isomers thereof.

15. A process for the preparation of compounds of formula I according to claim 1, characterised in that a compound of formula (II): 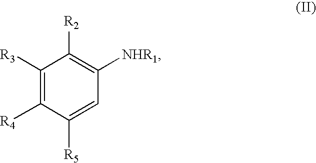
wherein R₁, R₂, R₃, R₄ and R₅ are as defined for formula (I),
is reacted with a compound of formula (III): 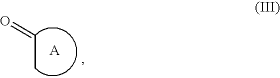
wherein 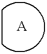
is as defined for formula (I),
and with a compound of formula (IV): 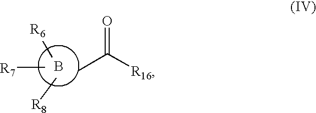
wherein 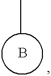
R₆, R₇, R₈ and R₁₆ are as defined for formula (I),
to yield the compound of formula (Ia), a particular case of the compounds of formula (I): 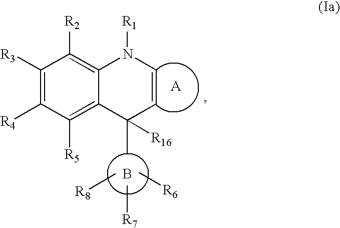
wherein 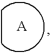
R₁, R₂, R₃, R₄, R₅, R₆, R₇, R₈, R₁₆ and 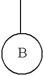
are as defined hereinbefore,
which, if desired, is reduced to yield the compound of formula (Ib), a particular case of the compounds of formula (I): 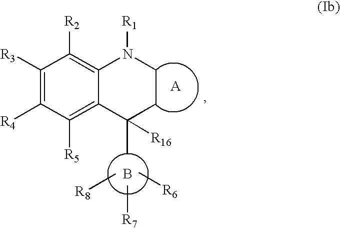
wherein 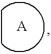
R₁, R₂, R₃, R₄, R₅, R₆, R₇, R₈, R₁₆ and 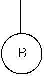
are as defined hereinbefore,
the compounds of formulae (Ia) and (Ib) constituting the totality of the compounds of formula (I), which are purified, if necessary, according to a conventional purification technique, are separated, if desired, into their optical isomers according to a conventional separation technique and are converted, if desired, into their addition salts with a pharmaceutically acceptable acid or base.

16. A pharmaceutical composition comprising as active ingredient a compound according to any one of claims 1 to 14 in combination with one or more inert, non-toxic and pharmaceutically acceptable carriers.

17. A pharmaceutical composition according to claim 16 for use as an anti-cancer medicament.
